(19) United States
(12) Patent Application Publication
(10) Pub. No.: US 2024/0335545 A1
(43) Pub. Date: Oct. 10, 2024

(54) Methods of Treating Graft Versus Host Disease

(71) Applicant: The Medical College of Wisconsin, Inc., Milwaukee, WI (US)

(72) Inventors: William R. Clarke, West Allis, WI (US); Brian F. Volkman, Muskego, WI (US); Francis C. Peterson, Racine, WI (US); Michael B. Dwinell, Milwaukee, WI (US); Chad A. Koplinski, Milwaukee, WI (US)

(21) Appl. No.: 18/627,930

(22) Filed: Apr. 5, 2024

Related U.S. Application Data
(60) Provisional application No. 63/494,950, filed on Apr. 7, 2023.

(51) Int. Cl. A61K 47/55; A61K 38/19; A61P 37/06

(52) U.S. Cl. CPC A61K 47/55; A61K 38/195; A61P 37/06

(57) ABSTRACT

Disclosed are methods of treating graft versus host disease (GVHD) or treating ocular graft versus host disease (oGVHD). The methods may comprise administering a therapeutically effective amount of a composition comprising CCL20 locked dimer to a subject, e.g., a subject suffering from GVHD. Also disclosed are modified CCL20LD compositions comprising an unstructured polypeptide.

### CROSS-REFERENCE TO RELATED APPLICATIONS

The present application claims priority to U.S. Provisional Patent Application No. 63/494,950 that was filed Apr. 7, 2023, the entire contents of which are hereby incorporated by reference.

### STATEMENT REGARDING FEDERALLY SPONSORED RESEARCH OR DEVELOPMENT

This invention was made with government support under HL164271 awarded by the National Institutes of Health. The government has certain rights in the invention.

### SEQUENCE LISTING

A Sequence Listing accompanies this application and is submitted as an xml file of the sequence listing named “650053_01052.xml” which is 2,945 bytes in size and was created on Feb. 16, 2024. The sequence listing is electronically submitted via Patent Center and is incorporated herein by reference in its entirety.

### BACKGROUND AND SUMMARY

Chronic graft versus host disease (cGVHD) is the leading cause of long-term morbidity and mortality following allogeneic hematopoietic stem cell transplantation. This devastating condition affects 20-50% of the ˜45,000 allogeneic bone marrow transplant recipients each year. It is now widely accepted that dysregulated T helper 17 (Th17) cells contribute to the development and progression of cGVHD. In a pathogenic process characteristic of Th17-mediated autoimmune disease, cGVHD can damage virtually any organ system. Prednisone, the standard treatment for over 30 years, itself causes significant issues, including weight gain, bone loss, diabetes, hypertension, mood swings, cataract formation, and increased risk of infection. Up to 40% of patients diagnosed with cGVHD die within seven years during treatment and half of cGVHD patients become unresponsive to corticosteroid therapy, with few alternatives. This poor clinical outlook highlights a clear unmet need for a safe and effective treatment for cGVHD.

### BRIEF DESCRIPTION OF THE FIGURES

FIG. 1. Schematic showing the vicious cycle of CCL20/Th17 inflammation hypothesized to be involved in GVHD.

FIG. 2. Development of CCL20LD. (A) Sequence of human CCL20 protein annotated with secondary structure and native disulfide pairing (cyan) and engineered disulfide (red). (B) Crystal structure of CCL20LD. (C) Treatment with CCL20LD prevents ear skin inflammation in a mouse model of human psoriasis.

FIG. 3. CCL20LD treatment reduces Th17 inflammation in skin and joints. B10.RIII mice were treated with PBS vehicle or 100 μg CCL20LD for 7 consecutive days or 100 ug anti-IL-17A antibodies every other day beginning at day 7 after IL23MC induction. (A) Representative photographs and images of H&E-stained ear sections. (B) Measured ear thickness showing a significant reduction upon treatment with CCL20LD but not anti-IL17A mAb. (C) Representative photographs and H&E sections of front paw. (D) Arthritic incidence severity score. All data presented as mean±SEM. * P<0.05, ** P<0.01. *** P<0.001, by using two-way ANOVA with Bonferroni's test compared to IL-23 MC+PBS group.

FIG. 4. Th17 infiltration and increased expression of CCL20 in the skin of mice with cGVHD. Lethally irradiated BALB/c mice were injected with 2.5 or 7.5×10⁶ T cell depleted bone marrow cells from wild-type or IgH^{μγ1} DBA/2 mice (in which B cells do not secrete antibodies) to induce cGVHD (WT), no disease (25M IgH^{μγ1}) or transient GVHD (25M IgH^{μγ1}). (A) Mean±SE of CCL20 expression in skin at 40 and 60 days after HCT. Relative gene expression levels were normalized to GAPDH. N=3 from triplicate experiments (** P<. 01, *** P<. 001). (B) Percentage of CCR6⁺IL17⁺ double-positive donor CD4⁺ T cells in PLNs at day 40 after HCT (mean±SE; 2 or more replicates of N=4.)

FIG. 5. CCL20LD PK. An estimate of the half-life in B10.RIII mice (T_{1/2}˜0.5 h) was obtained by exponential fitting of the decline in serum concentration (measured by CCL20 ELISA) after injection of 400 ug CCL20LD.

FIG. 6 shows a reduction of IL17⁺, IFN-γ⁺ T cells in the liver in a minor mismatch (H2Kb-B6 H2Kb-C3h.sw) murine bone marrow transplant model of chronic GVHD.

FIGS. 7A, 7B, and 7C show a reduction of IL17⁺, IFN-γ⁺ T cells in the (A) lung, (B) spleen, and (C) skin in a minor mismatch (H2Kb-B6 H2Kb-C3h.sw) murine bone marrow transplant model of chronic GVHD.

FIG. 8 shows a survival curve demonstrating a trend towards increased survival of mice administered CCL20LD, as compared to vehicle, in a mouse model of cGVHD. “HCT” is an abbreviation for hematopoietic stem cell transplantation.

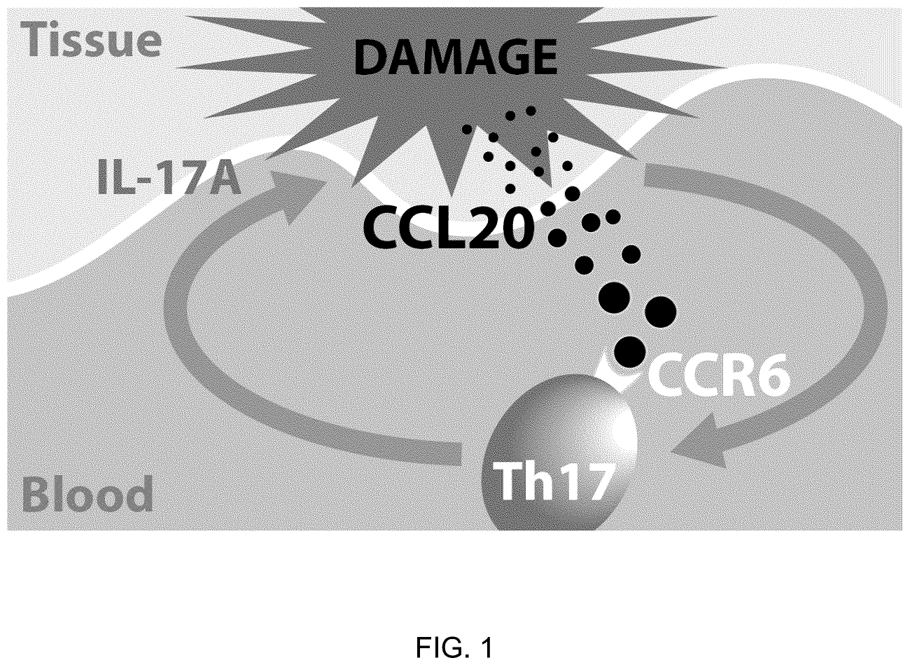

### DETAILED DESCRIPTION

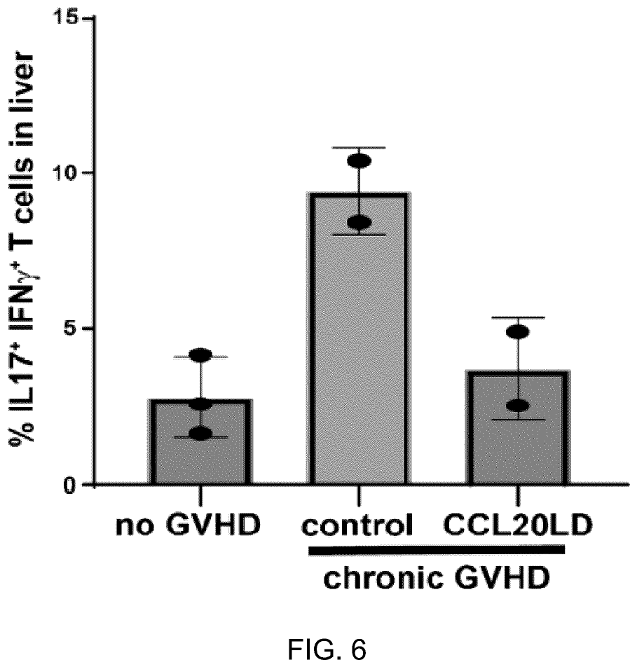

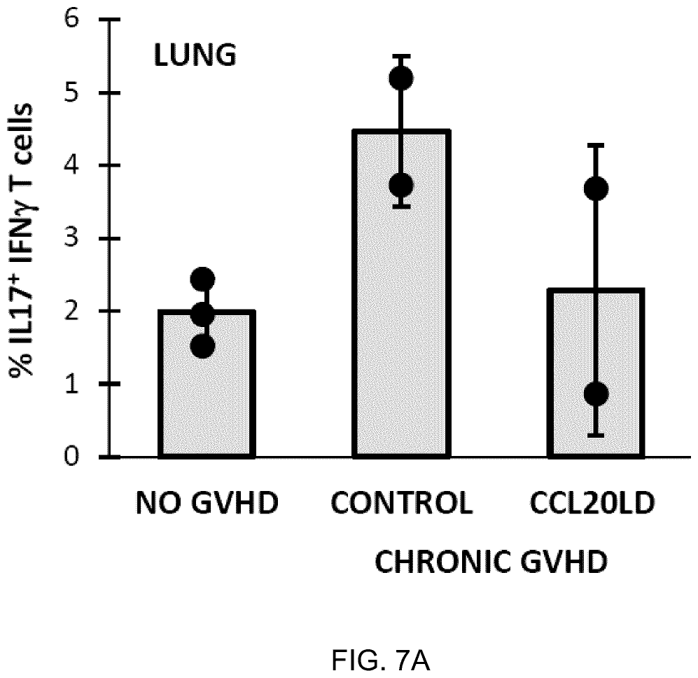

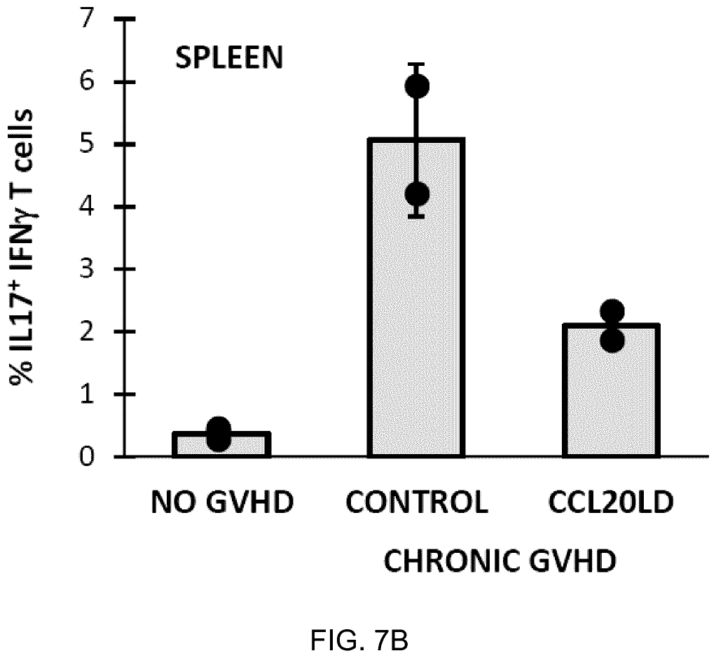

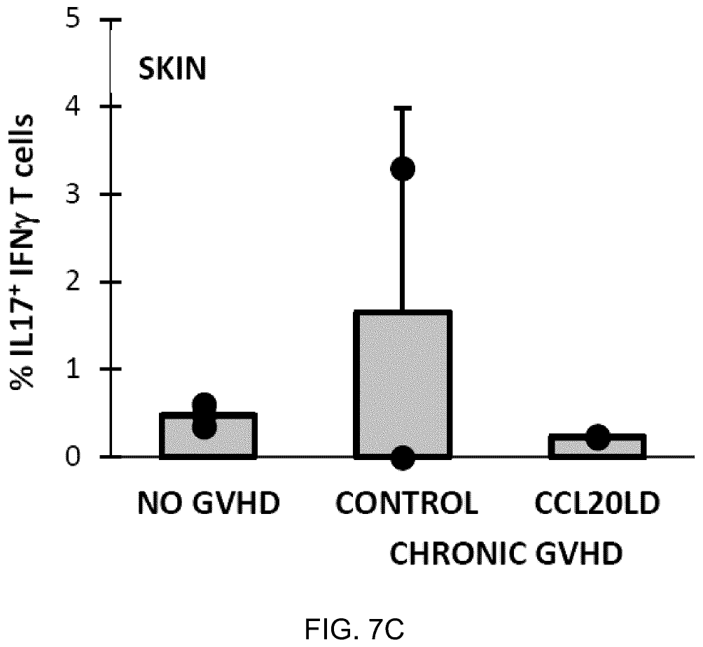

The inventors have discovered that a composition comprising homodimeric human chemokine motif C-C ligand 20 (CCL20) with a serine to cysteine mutation at position 64, with reference to SEQ ID NO: 2, may be a novel treatment for graft versus host disease (GVHD), a disease with a serious unmet therapeutic need and potentially life-threatening consequences. The inventors have discovered that CCL20LD is effective in reducing the frequency of pathogenic Th17 T cells in an animal model of GVHD (FIGS. 6 and 7A-7C). Unexpectedly, CCL20LD reduces the frequency of IL-17⁺, IFN-γ⁺ T cells in the liver to, essentially, the same as healthy control animals. There are currently no targeted therapies for the treatment of cGVHD. As discussed below, the current treatments available to subjects suffering from GVHD come at a potential cost of lack of durable efficacy and a risk of severe side-effects.

In addition, the inventors are unaware of another therapy which has been demonstrated to effectively modulate TH17 cells.

Graft-versus-host disease (GVHD) is a complication of allogeneic stem cell transplantation and reduced-intensity allogeneic stem cell transplantation. During allogeneic stem cell transplantation, a patient receives stem cells from a donor or donated umbilical cord blood. GVHD occurs when the donor's T cells (the graft) view the patient's healthy cells (the host) as foreign, and attack and damage them. Graft-versus-host disease can be mild, moderate, or severe. In some cases, it can be life-threatening. The risk and severity of this immune-mediated condition are directly related to the degree of mismatch between a host and the donor of hematopoietic cells. For example, GVHD develops in up to 30% of recipients of human leukocyte antigen (HLA)-matched sibling marrow, in up to 60% of recipients of HLA-matched unrelated donor marrow, and in a higher percentage of recipient of HLA-mismatched marrow.

As used herein, “human leukocyte antigen (HLA)” refers to the human version of the major histocompatibility complex (MHC), proteins which present antigens on the surface of a cell that may be recognized by T lymphocytes. The major HLA genes are HLA-A, -B, and -C which correspond to MHC class I, and HLA-DP, -DM, -DO, -DQ and -DR correspond to MHC class II. Typing of a recipient and donor's HLAs is an aspect of selecting an appropriate donor for allogeneic stem cell transplantation. In some cases, donors with HLA mismatches to the recipient, i.e., the donor and the recipient do not have the same HLA alleles, are selected which increases the likelihood of the recipient developing GVHD. Methods of determining HLA matching are known in the art.

Unless the patient's donor is an identical twin, a patient receiving an allogeneic stem cell transplant will typically receive some type of GVHD prevention. This may include removing T cells from the donor graft and/or giving medications to suppress the T cells in the graft so that they do not attack the patient's cells. Different combinations of medications may be administered. Some common medications that are given to prevent GVHD include, abatacept (Orencia®), antithymocyte globulin (ATG), alemtuzumab (Campath®), Belumosudil (Rezurock™), corticosteroids, e.g., methylprednisolone or prednisone, cyclophosphamide (Cytoxan®), cyclosporine, methotrexate (Trexall®), mycophenolate mofetil (CellCept®), the ROCK2 inhibitor ruxolitinib, sirolimus (Rapamune®), and tacrolimus (Prograf®). There are two main categories of GVHD: acute graft-versus-host disease and chronic graft-versus-host disease. Each type affects different organs and tissues and has different signs and symptoms. Patients receiving an allogeneic stem cell transplant may develop one type, both types, or neither type.

Acute GVHD (aGVHD)

This usually develops within the first 100 days after transplantation, e.g., less than 100 days, less than 90 days, less than 80 days, less than 70 days, less than 60 days, less than 50 days, less than 40 days, less than 30 days, less than 20 days, less than 10 days, but it can occur later. Acute GVHD can affect the skin, the gastrointestinal tract or the liver. Symptoms may include: a rash, with burning and redness of the skin, nausea, vomiting, abdominal cramps, loss of appetite, diarrhea, or jaundice. Many patients who develop acute GVHD are successfully treated with increased immunosuppression in the form of corticosteroids (medicines such as prednisone, methylprednisolone, dexamethasone, beclomethasone and budesonide). Patients with mild skin-only acute GVHD will typically continue with their original medications such as cyclosporine or tacrolimus and add a topical steroid cream (topical means applied directly to a part of the body) to their treatment plan. Treatment for patients with systemic or “whole-system” manifestations and/or more severe acute GVHD usually consists of continuing the original immunosuppressive prevention and adding a corticosteroid such as methylprednisolone or prednisone.

Chronic GVHD (cGVHD)

cGVHD is a syndrome of variable clinical features that resembles autoimmune and other immunologic disorders (eg, scleroderma, Sjögren's syndrome, primary biliary cirrhosis, bronchiolitis obliterans). Clinical manifestations may be widespread or they may be restricted to a single organ or site. The primary clinical manifestations are skin involvement (resembling lichen planus or cutaneous scleroderma), dry oral mucosa, gastrointestinal tract ulcerations and sclerosis, elevated serum bilirubin, and bronchiolitis obliterans. In addition, ocular issues may also manifest. By contrast, patients with aGVHD commonly demonstrate a maculopapular rash, abdominal cramps with diarrhea, and elevated serum bilirubin.

Symptoms of chronic GVHD of the skin include reddened areas or discoloration of the skin and raised, cellulite-like, or thickened areas of the skin that are hard to pinch. Symptoms of chronic oral GVHD include mouth sores, dry mouth, or white patches inside the mouth. Chronic oral GVHD can also cause pain or sensitivity in the mouth, particularly with certain foods. Symptoms of chronic GVHD of the gut or liver include difficulty swallowing, loss of appetite, nausea, vomiting, or abnormal liver functioning.

Typically, the severe effects of ocular GVHD appear during the chronic phase of the disease. Therapies for chronic ocular GVHD are limited. Ocular steroids are used acutely but long-term use causes glaucoma and cataracts. Ocular GVHD presents as severe dry eye disease due to the immunologic damage caused to the meibomian glands that produce the protective tears for the anterior eye. Thus, ocular GVHD is also termed ocular dry eye syndrome (oDES). Use of ‘artificial tears’ can be beneficial but requires application several times an hour and is impractical at night.

In a longitudinal study of patients after allogeneic bone marrow transplant 52% had ocular DES that caused moderate to significant damage to vision. See Wang J. et al. “Risk factors for the development of ocular graft-versus-host disease (GVHD) dry eye syndrome in patients with chronic GVHD” Br J Ophthalmol. 2015 November; 99 (11): 1514-8, (PMID 25947556), which is incorporated by reference herein in its entirety. Therefore, oDES is a serious clinical problem for which there are no effective long-term treatments available.

Chronic GVHD usually develops more than 3 months, e.g., 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, or greater than 36 months after an allogeneic stem cell transplant and is one of the leading causes of medical problems and death after an allogeneic stem cell transplantation. Chronic GVHD may cause, for example, skin rash or reddened areas on the skin, raised skin, blistering, thickening or tightening of the skin, yellow discoloration of the skin and/or eyes, abnormal blood test results, nausea, vomiting, diarrhea, abdominal swelling, abdominal cramping, increased dryness or irritation of the eyes, vision changes, dry mouth, white patches inside the mouth, pain or sensitivity to spicy foods, shortness of breath, difficulty swallowing, pain with swallowing, weight loss, fatigue, muscle weakness, muscle pain, increased urinary frequency, burning or bleeding with urination, vaginal dryness or tightening, or penile dysfunction.

### Methods of Treating Graft Versus Host Disease (GVHD) Using CCL20 Locked Dimer (CCL20LD)

Accordingly, in an aspect of the current disclosure, methods of treating graft versus host disease (GVHD) are provided. In some embodiments, the methods comprise administering a therapeutically effective amount of a composition comprising a CCL20 locked dimer to a subject in need thereof.

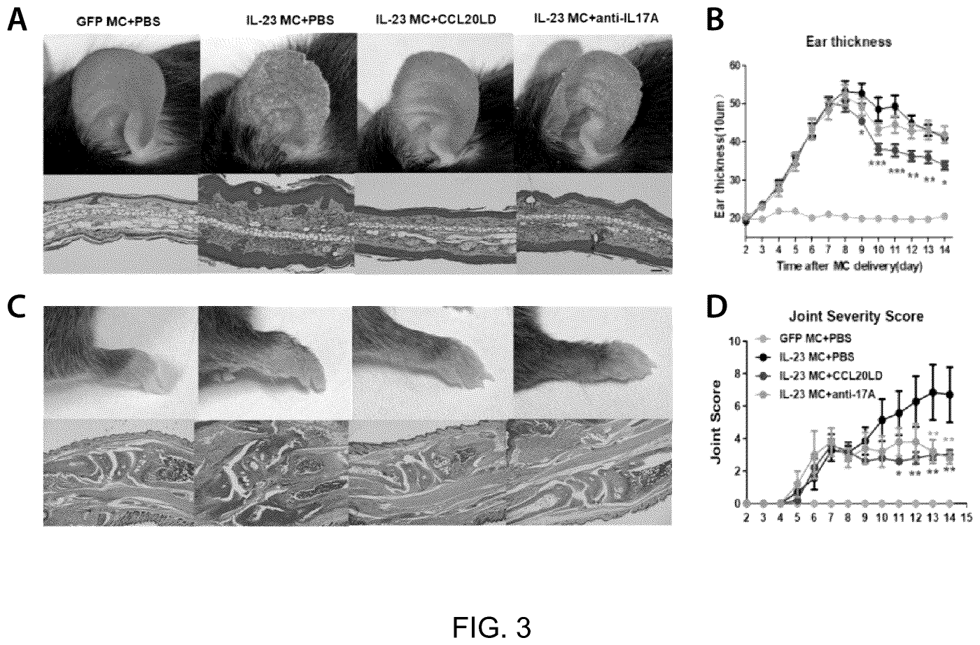

Previously, the inventors discovered that a serine to cysteine substitution at position 64, with reference to SEQ ID NO: 2, of human protein chemokine motif C-C ligand 20 (CCL20) resulted in a novel composition that formed a stable covalently linked homodimer. See, U.S. Pat. No. 10,738,095, which is incorporated by reference herein in its entirety. Furthermore, the inventors have demonstrated that CCL20LD is effective in treating psoriasis in animal models. See, e.g., FIG. 3 and U.S. Pat. Pub. No. 20200360479, which is incorporated by reference herein in its entirety.

As used herein, “CCL20 locked dimer” or “CCL20LD”, used interchangeably, refer to a dimer of human CCL20 protein with a serine to cysteine substitution at position 64, with reference to SEQ ID NO: 2, in at least one of the two CCL20 proteins making up the dimer. The CCL20LD may comprise one member of the dimer with a sequence comprising SEQ ID NO: 1, or a sequence with at least 95% identity to SEQ ID NO: 1 and comprising a cysteine at position 64, or both members of the dimer may comprise SEQ ID NO: 1, or a sequence with at least 95% identity to SEQ ID NO: 1 and comprising a cysteine at position 64. In addition, one member of the dimer making up the CCL20LD may consist of SEQ ID NO: 1, or a sequence with at least 95% identity to SEQ ID NO: 1, or both members of the dimer, i.e., CCL20LD, may consist of SEQ ID NO: 1, or a sequence with at least 95% identity to SEQ ID NO: 1 and comprising a cysteine at position 64.

The CCL20LD compositions may be produced according to methods known in the art which are, for example, disclosed in U.S. Pat. No. 10,738,095. Briefly, E. coli cells expressing the CCL20LD compositions may be lysed by French Press and centrifuged for 20 minutes at 15,000 rpm. The insoluble fraction may be resuspended in a suitable buffer, e.g., 6M guanidine buffer, and purified by nickel chromatography. CCL20S64C elutions may be pooled and refolded dropwise into a suitable buffer, e.g., a 20 mM cysteine, 0.5 mM cystine, 100 mM Tris, (pH 8.0) buffer. CCL20WT elutions may be refolded in a 10 mM cysteine, 0.5 mM cystine, 100 mM Tris (pH 8.0) buffer. Elutions may be HPLC purified and lyophilized. Purity and molecular weights may be confirmed by, e.g., SDS-PAGE and MALDI-TOF spectrometry.

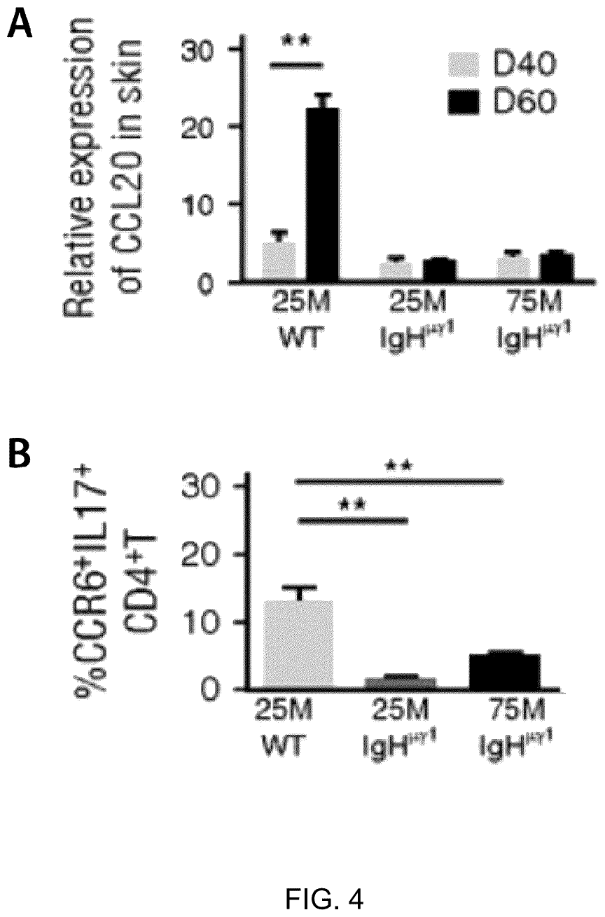

Referring now to FIG. 4, the inventors have demonstrated that CCL20 expression is increased in skin samples in a mouse model of GVHD (FIG. 4A). In addition, the inventors have demonstrated that there is an increase in CD4⁺, CCR6⁺, IL-17⁺ T cells (Th17 cells expressing CCR6, the ligand for CCL20) in peripheral lymph nodes in the same model of GVHD. These data support the hypothesis that Th17 cells expressing CCR6 may be responsible for the signs and symptoms associated with GVHD and that CCL20LD may be effective in preventing those signs and symptoms caused by Th17 cells migrating to affected tissues based on the presence of CCL20.

Referring now to FIG. 6, the inventors have demonstrated that administration of CCL20LD in an animal model of cGVHD reduced the frequency of IL17⁺, IFN-γ⁺ T cells, which are likely pathogenic, in the liver.

Referring to FIGS. 7A, 7B, and 7C, the inventors have demonstrated that administration of CCL20LD in an animal model of cGVHD reduced the frequency of IL17⁺, IFN-γ⁺ T cells in the lung (FIG. 7A), spleen (FIG. 7B), and skin (FIG. 7C).

Therefore, the data disclosed herein demonstrates that CCL20LD is unexpectedly effective in reducing the frequency of pathogenic Th17 cells in an animal model of GVHD.

As used herein, “treat,” “treating,” and grammatical variations thereof, refer to the management and care of a subject for the purpose of combating the disease, condition, or disorder. The terms embrace both preventative, i.e., prophylactic, and palliative treatments. Treating includes the administration of a composition of the present invention to prevent, ameliorate and/or improve the onset of the symptoms or complications, alleviating the symptoms or complications, or eliminating the disease, condition, or disorder, i.e., GVHD.

As used herein, “administering” refers to any means for introducing the CCL20 locked dimer polypeptide into the body of a subject. Exemplary routes include but are not limited to oral, buccal, sublingual, pulmonary, transdermal, transmucosal, as well as subcutaneous, intraperitoneal, intravenous, topical ocular administration alone or in suitable carrier formulation to prolong ocular residence, subconjunctival, and intramuscular routes.

As used herein, “therapeutically effective amount” refers to the amount or dose of the composition, i.e., CCL20LD, upon single or multiple dose administration to a subject, which provides the desired effect in the subject under diagnosis or treatment. The disclosed methods may include administering an effective amount of the disclosed compositions (e.g., as present in a pharmaceutical composition) for treating GVHD, e.g., for reducing a sign or symptom related to GVHD, which are discussed in detail below.

A subject suffering from GVHD may exhibit one or more symptoms of oral GVHD. Exemplary symptoms of oral GVHD include, but are not limited to, oral tissue inflammation, dry mouth, punctate or generalized mucosal erythema, white striae or papules on the oral mucosa and lips, mucosal erosion-desquamation-ulceration, pain or sensitivity to spicy foods, difficulty swallowing, pain with swallowing, pharyngo-esophageal stricture, xerostomia, lichen planus, poor bolus control, pharyngeal retention, excessive mucous secretion, oral tissue inflammation, and ulceration. A subject in need therefore may suffer from refractory GVHD in which steroids, the mTOR inhibitor cyclosporin, ROCK2 inhibitors, or other commonly used immunosuppressant therapies are insufficient to control the GVHD. Allogeneic hematopoietic cell transplantation is an effective therapy for the treatment of many hematologic malignancies, including, for example, B-cell and T-cell malignancies. In allogeneic hematopoietic cell transplantation, bone marrow (or, in some cases, peripheral blood) from an unrelated or a related (but not identical twin) donor is used to replace the healthy blood cells destroyed in the cancer patient. The bone marrow (or peripheral blood) contains stem cells, which are the precursors to all the different cell types (e.g., red cells, phagocytes, platelets and lymphocytes) found in blood.

GVHD is an inflammatory disease initiated by T cells in the donor graft that recognize histocompatibility and other tissue antigens of the host and GVHD is mediated by a variety of effector cells and inflammatory cytokines. GVHD presents in both acute and chronic forms. The most common symptomatic organs are the skin, liver, and gastrointestinal tract, including the oral cavity and oropharyngeal regions. GVHD may involve other organs such as the lung. Treatment of GVHD is generally only 50-75% successful; the remainder of patients generally do not survive. The risk and severity of this immune-mediated condition are directly related to the degree of mismatch between a host and the donor of hematopoietic cells. For example, GVHD develops in up to 30% of recipients of human leukocyte antigen (HLA)-matched sibling marrow, in up to 60% of recipients of HLA-matched unrelated donor marrow, and in a higher percentage of recipient of HLA-mismatched marrow.

Patients with mild intestinal GVHD present with anorexia, nausea, vomiting, abdominal pain and diarrhea, whereas patients with severe GVHD are disabled by these symptoms. If untreated, symptoms of intestinal GVHD persist and often progress; spontaneous remissions are unusual. In its most severe form, GVHD leads to necrosis and exfoliation of most of the epithelial cells of the intestinal mucosa, a frequently fatal condition.

The symptoms of acute GVHD usually present within 100 days of transplantation. The symptoms of chronic GVHD usually present somewhat later, up to three years after allogeneic HCT, and are often proceeded by a history of acute GVHD. Oral manifestations of GVHD are seen in both acute GVHD (aGVHD) and chronic GVHD (cGVHD). Oral involvement ranges between 33% and 75% for patients with aGVHD and up to about 80% for those with cGVHD. Involvement of the salivary glands may cause dryness of the oral mucosa and oral pain may be the first presenting symptom. Oral lesions in GVHD may be lichenoid or lupus-like in appearance. Oral findings of aGVHD include painful desquamative, erythematous, and ulcerative mucosal lesions. In cGVHD, they are lichenoid with associated erythema and ulcerations; additionally, they may be associated with sicca syndrome characterized by xerostomia and progressive salivary gland atrophy. Oral complications include pain due to the mucosal changes, altered or reduced taste, and may have a potential impact on speech, deglutition, and use of oral prostheses (when present). Oral infection, particularly due to Candida species, and dental demineralization and caries may also occur. Oral manifestations of cGVHD can significantly affect the life quality of patients through discomfort and impairment of the oral intake leading to malnutrition and increased morbidity. The conventional management of oral cGVHD consists of systemic immunosuppressive therapies combined with proper oral hygiene and the judicious use of topical steroids. However, for patients with oral cGVHD as the most significant clinical finding, the use of systemic immunosuppressants may result in immunosuppression of the host with attendant systemic complications. In addition, some patients experience considerable and refractory oral complications, even with maximum doses of systemic immunosuppressants. First-line therapy of oral GVHD is mostly systemic in nature, consisting of cyclosporin and steroids. The most common salvage treatments for cGVHD are thalidomide, tacrolimus, mycophenolate mofetil, T cell depletion by Campath-1, and phototherapy. Oral GVHD is often refractory to conventional treatment and therefore complementary topical treatment is required. Several agents are currently used for local treatments such as palliative rinses, topical immunosuppressive agents, thalidomide, retinoids, and phototherapy for oral GVHD.

In some cases, subjects are considered “steroid refractory” or to have “steroid refractory GVHD” which, as used herein, refers to GVHD, e.g., cGVHD, progression while on prednisone at ≥1 mg/kg/day for 1-2 weeks, or stable cGVHD while on ≥0.5 mg/kg/day for 1-2 months.

In addition to the above-described signs and symptoms of GVHD, ocular GVHD (oGVHD) has emerged as a major, and under-appreciated, complication of allogeneic stem cell transplantation. oGVHD may cause meibomian gland obstruction, anterior and posterior blepharitis, associated scarring of the lacrimal gland leading to decreased tear production, and conjunctival hyperemia with pseudomembrane and membrane formation. Chronic inflammation can also lead to conjunctival necrosis and cicatricial scarring and fibrosis. Patients with oGVHD often have corneal manifestations including punctate epithelial keratopathy and filamentary keratitis. Severe disease can lead to corneal erosions, thinning, ulcerations and possible perforations. In addition, patients with oGVHD may experience the following symptoms: dry eye, foreign body sensation, redness, epiphora, photophobia, blurred vision, and eye irritation. Keratoconjunctivitis sicca (KCS) is the most common manifestation of oGVHD and can be exacerbated by concomitant radiation and chemotherapy. Furthermore, KCS can last years after the resolution of other systemic manifestations of cGVHD.

Current methods to treat ocular symptoms of GVHD, e.g., topical steroids, may be ineffective. See, for example, Yin et al. “Reduced Efficacy of Low-dose Topical Steroids in Dry Eye Disease Associated with Graft-versus-host Disease” Am J Ophthalmol. 2018 June; 190:17-23, which is incorporated herein by reference. Surprisingly, while low dose topical loteprednol, a corticosteroid, was effective in reducing symptoms of dry eye disease (DED) in subjects without history of hematopoietic stem cell transplant, the same treatment did not produce a significant difference in the DED symptoms of subjects who had undergone hematopoietic stem cell transplant. Accordingly, oGVHD is difficult to treat with existing medications and, furthermore, it is unpredictable which treatments may be effective for treating oGVHD.

In contrast to traditional therapies, the disclosed compositions and methods comprising CCL20LD may be effective in the treatment of oGVHD because CCL20LD may directly address the underlying pathology of oGVHD by preventing migration of Th17 cells to the eye. Accordingly, the disclosed methods, e.g., “treating oGVHD” may result in the reduction or elimination of one or more symptoms of oGVHD as discussed above. Treatment of oGVHD may be provided by administration of CCL20LD by a systemic or more limited route, e.g., intravenous, oral, or topical administration to the eye.

Exemplary ranges for therapeutically or prophylactically effective amounts of CCL20 locked dimer polypeptide may be 0.1 nM-0.1M, 0.1 nM-0.05M, 0.05 nM-15 μM, and 0.01 nM-10 μM, e.g., as a concentration in a relevant tissue or in the bloodstream. A therapeutically effective or prophylactically effective dose of the disclosed CCL20LD compositions may comprise 1 ng, 10 ng, 100 ng, 1 ug, 10 ug, 100 ug, 1 mg, 10 mg, 100 mg, 1 g, 10 g, greater than 10 g, or any value between 1 ng-10 g. Alternatively, dosing may be considered in a mg/kg/day manner and systemic dosages may be range from 0.01 to 100 mg/kg/day. It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Frequency of topical application, e.g., ophthalmic topical administration, to a subject (for example, a human or animal) may vary between subjects. For example, in some circumstances, the disclosed formulations may be applied as frequently as once every ten minutes or as infrequently as once every day. The formulation may be applied once, twice, three times or more over a period of one day, two days, three days, four days, five days, or more than five days. Where an extended-release vehicle is included in the formulation, the formulation may be topically applied with a frequency of at least one time a day to one time a week, or one time a month to one time every six months. Frequency of topical application can be determined by numerous considerations, including level of symptom relief provided, pain relief experienced, as well as other pertinent health considerations, such as possible drug interactions. The disclosed formulations may be administered using any desired technique. For example, in some cases, the formulations may be eye drops that are administered manually by a user. In other cases, the formulations may be topically applied as a gel or ointment directly to a desired region of the eye using a swab or other type of applicator. In some embodiments, the disclosed formulations may be delivered using a device designed for immediate formulation release or extended formulation release. For example, the formulation may be delivered using one or more of the following devices: external pumps, contact lenses, punctal plugs, muco-adhesive tablets, pills, capsules, pellets, particles, plasters, an ocular insert device, strips placed onto the conjunctiva or cornea, conjunctival inserts or depots, subconjunctival, subtenon, and intravitreal injections, or another suitable type of device. The CCL20LD may be administered via the subconjunctival route. Subconjunctival administration of the CCL20LD may be performed once a week, or more, or once a month, or more, at the direction of a physician.

The amount of CCL20 locked dimer polypeptide in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for case of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention is dictated by and directly dependent on (a) the unique characteristics of the active compound and the particular therapeutic effect to be achieved, and (b) the limitations inherent in the art of compounding such as active compound for the treatment of sensitivity in individuals.

The CCL20 locked dimer polypeptide of the present invention, optionally comprising other pharmaceutically active compounds, can be administered to a patient orally, rectally, parenterally, (e.g., intravenously, intramuscularly, or subcutaneously) intracisternally, intravaginally, intraperitoneally, intravesically, locally (for example, powders, ointments or drops), or as an ophthalmic topical solution or ointment or as a buccal or nasal spray. Other contemplated formulations include projected nanoparticles, liposomal preparations, rescaled erythrocytes containing the active ingredient, immunologically-based formulations and topical ophthalmic formulations in sterile petrolatum or other excipient agents to prolong external ocular residence time of the CCL20LD composition.

The disclosed formulations, e.g., ophthalmic formulations, may also include an extended-release vehicle, such as a biocompatible polymer, dissolved in the carrier or by itself impregnated with CCL20LD to hold the CCL20LD and slowly release it into the tear film or onto the ocular surface, preferably for an extended-release period, e.g., one day, two days, three days, four days, five days, six days, seven days, or more. The biocompatible polymer may be biodegradable or non-biodegradable, depending on desired use and application schedule. Example biocompatible polymers that may be used in the disclosed formulations as an extended-release vehicle include but are not limited to poly-2-hydroxyethylmethacrylate (p-HEMA hydrogels), poly(lactic-co-glycolic) acid (PLGA), polycaprolactone (PCL), hydroxypropyl cellulose, Anecortave acetate (AnA), gelatin, and/or collagen. The inclusion of an extended-release vehicle may, in some cases, allow for less frequent application while still providing relief from oGVHD symptoms.

Parenteral administration of a pharmaceutical composition includes any route of administration characterized by physical breaching of a tissue of a human and administration of the pharmaceutical composition through the breach in the tissue. Parenteral administration thus includes administration of a pharmaceutical composition by injection of the composition, by application of the composition through a surgical incision, by application of the composition through a tissue-penetrating non-surgical wound, and the like. In particular, parenteral administration includes subcutaneous, intraperitoneal, intravenous, intraarterial, intramuscular, or intrasternal injection and intravenous, intraarterial, or kidney dialytic infusion techniques.

Compositions suitable for parenteral injection comprise the CCL20 locked dimer of the invention combined with a pharmaceutically acceptable carrier such as physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, isotonic saline, ethanol, polyols (e.g., propylene glycol, polyethylene glycol, glycerol, and the like), suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants. Such formulations can be prepared, packaged, or sold in a form suitable for bolus administration or for continuous administration. Injectable formulations can be prepared, packaged, or sold in unit dosage form, such as in ampules, in multi-dose containers containing a preservative, or in single-use devices for auto-injection or injection by a medical practitioner.

Formulations for parenteral administration include suspensions, solutions, emulsions in oily or aqueous vehicles, pastes, and implantable sustained-release or biodegradable formulations. Such formulations can further comprise one or more additional ingredients including suspending, stabilizing, or dispersing agents. In one embodiment of a formulation for parenteral administration, the CCL20 locked dimer polypeptide is provided in dry (i.e., powder or granular) form for reconstitution with a suitable vehicle (e.g., sterile pyrogen-free water or sterile buffered salt solution) prior to parenteral administration of the reconstituted composition.

The pharmaceutical compositions can be prepared, packaged, or sold in the form of a sterile injectable aqueous or oily suspension or solution. This suspension or solution can be formulated according to the known art. Such sterile injectable formulations can be prepared using a non-toxic parenterally-acceptable diluent or solvent, such as water or 1,3-butanediol, for example. Other acceptable diluents and solvents include Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation can comprise pharmaceutically acceptable polymeric or hydrophobic materials such as an emulsion, an ion exchange resin, a sparingly soluble polymer, or a sparingly soluble salt. Exemplary formulations for the disclosed pharmaceutical compositions may comprise compositions suitable for ocular administration, e.g., topical ophthalmic administration.

The CCL20 locked dimer polypeptide of the present invention may also contain adjuvants such as suspending, preserving, wetting, emulsifying, and/or dispersing agents, including, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, such as aluminum monostearate and/or gelatin.

The disclosed formulations may also include one or more additives. Additives that may be included in the disclosed formulations include but are not limited to demulcents, preservatives, emollients, ionic species, pH-adjusting agents, and other possible additives. Example demulcents that may be used in the disclosed formulations include glycerin, carboxymethylcellulose, hydroxyethyl cellulose, hydroxypropyl methylcellulose, methylcellulose, dextran 70, gelatin, polyethylene glycol 300, polyethylene glycol 400, polysorbate 80, polyvinyl alcohol, povidone, etc. Examples of preservatives that may be used in the disclosed formulations include benzalkonium chloride and other known preservatives, such as chlorobutanol. Emollients may include lanolin preparations, various mineral oils, Omega 3, paraffin, petroleum, and waxes. Zinc and or sodium ions may be included in the disclosed formulation as an ionic species, in some embodiments. Possible pH-adjusting agents that may be used include citrate buffers (e.g., sodium citrate), borate buffers (e.g., sodium borate), and other acidic or basic compounds. Numerous configurations and variations of additives may be used in the disclosed formulations.

Dosage forms can include solid or injectable implants or depots. In preferred embodiments, the implant comprises an effective amount of the locked dimer polypeptide and a biodegradable polymer. In preferred embodiments, a suitable biodegradable polymer can be selected from the group consisting of a polyaspartate, polyglutamate, poly(L-lactide), a poly(D,L-lactide), a poly(lactide-co-glycolide), a poly(ε-caprolactone), a polyanhydride, a poly(beta-hydroxy butyrate), a poly(ortho ester) and a polyphosphazene. In other embodiments, the implant comprises an effective amount of CCL20 locked dimer polypeptide and a silastic polymer. The implant provides the release of an effective amount of CCL20 locked dimer polypeptide for an extended period ranging from about one week to several years.

Solid dosage forms for oral administration include capsules, tablets, powders, and granules. In such solid dosage forms, the CCL20 locked dimer polypeptide is admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (c) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

A tablet comprising the active ingredient can, for example, be made by compressing or molding the active ingredient, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets can be made by molding, in a suitable device, a mixture of the active ingredient, a pharmaceutically acceptable carrier, and at least sufficient liquid to moisten the mixture.

Tablets may be manufactured with pharmaceutically acceptable excipients such as inert diluents, granulating and disintegrating agents, binding agents, and lubricating agents. Known dispersing agents include potato starch and sodium starch glycolate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets can be non-coated or coated using known methods to achieve delayed disintegration in the gastrointestinal tract of a human, thereby providing sustained release and absorption of the active ingredient. By way of example, a material such as glyceryl monostearate or glyceryl distearate can be used to coat tablets. Further by way of example, tablets can be coated using methods described in U.S. Pat. Nos. 4,256,108; 4,160,452; and U.S. Pat. No. 4,265,874 to form osmotically-controlled release tablets. Tablets can further comprise a sweetening agent, a flavoring agent, a coloring agent, a preservative, or some combination of these in order to provide pharmaceutically elegant and palatable preparation.

Solid dosage forms such as tablets, dragees, capsules, and granules can be prepared with coatings or shells, such as enteric coatings and others well known in the art. They may also contain opacifying agents and can also be of such composition that they release the active compound or compounds in a delayed manner. Examples of embedding compositions that can be used are polymeric substances and waxes. The active compounds can also be in micro-encapsulated form, if appropriate, with one or more of the above-mentioned excipients.

Solid compositions of a similar type may also be used as fillers in soft or hard filled gelatin capsules using such excipients as lactose or milk sugar, as well as high molecular weight polyethylene glycols, and the like. Hard capsules comprising the active ingredient can be made using a physiologically degradable composition, such as gelatin. Such hard capsules comprise the active ingredient, and can further comprise additional ingredients including, for example, an inert solid diluent such as calcium carbonate, calcium phosphate, or kaolin. Soft gelatin capsules comprising the active ingredient can be made using a physiologically degradable composition, such as gelatin. Such soft capsules comprise the active ingredient, which can be mixed with water or an oil medium such as peanut oil, liquid paraffin, or olive oil.

The present invention is further described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### Illustrative Embodiments

1. A method comprising: administering a therapeutically effective amount of a composition comprising a CCL20 locked dimer to a subject in need thereof, wherein the subject in need thereof has been diagnosed with or is suspected of having graft versus host disease (GVHD).

2. A method of treating graft versus host disease (GVHD) in a subject in need thereof, the method comprising: administering a therapeutically effective amount of a composition comprising a CCL20 locked dimer to the subject to treat GVHD in the subject.

3. The method of any one of embodiments 1 or 2, wherein the subject is suffering from ocular GVHD (oGVHD).

4. The method of any one of embodiments 1 or 2, wherein the subject is suffering from chronic GVHD (cGVHD).

5. The method of any one of embodiments 1-4, wherein the subject has previously failed steroid treatments or is considered steroid refractory.

6. The method of any one of embodiments 1-5, wherein the CCL20 locked dimer comprises two CCL20 monomers, wherein at least one of the CCL20 monomers comprises SEQ ID NO: 1.

7. The method of embodiment 6, wherein the CCL20 locked dimer comprises two monomers covalently linked together, wherein each monomer comprises SEQ ID NO: 1 or an amino acid sequence with at least a 95% sequence identity to SEQ ID NO: 1 and a S64C mutation relative to SEQ ID NO:1.

8. The method of embodiment 7, wherein each monomer comprises SEQ ID NO: 1.

9. The method of embodiment 9, wherein each monomer consists of SEQ ID NO: 1.

10. The method of any one of embodiments 1-9, wherein the method reduces a sign or symptom related to graft versus host disease (GVHD) selected from: anorexia, nausea, vomiting, abdominal pain and diarrhea, jaundice, oral tissue inflammation, dry mouth, punctate or generalized mucosal erythema, white striae or papules on the oral mucosa and lips, mucosal erosion-desquamation-ulceration, pain or sensitivity to spicy foods, difficulty swallowing, pain with swallowing, pharyngo-esophageal stricture, xerostomia, lichen planus, poor bolus control, pharyngeal retention, excessive mucous secretion, oral tissue inflammation, and ulceration.

11. A method of reducing a sign or symptom related to graft versus host disease (GVHD) in a subject suffering from GVHD, the method comprising: administering a therapeutically effective amount of a composition comprising a CCL20 locked dimer to the subject to reduce a sign or symptom related to graft versus host disease (GVHD).

12. The method of embodiment 11, wherein the sign or symptom related to graft versus host disease (GVHD) is one or more of: anorexia, nausea, vomiting, abdominal pain and diarrhea, jaundice, oral tissue inflammation, dry mouth, punctate or generalized mucosal erythema, white striae or papules on the oral mucosa and lips, mucosal erosion-desquamation-ulceration, pain or sensitivity to spicy foods, difficulty swallowing, pain with swallowing, pharyngo-esophageal stricture, xerostomia, lichen planus, poor bolus control, pharyngeal retention, excessive mucous secretion, oral tissue inflammation, and ulceration.

13. The method of any one of embodiments 11-12, wherein the subject is suffering from ocular GVHD (oGVHD).

14. The method of any one of embodiments 11-12, wherein the subject is suffering from chronic GVHD (cGVHD).

15. The method of any one of embodiments 11-14, wherein the subject has previously failed steroid treatments or is considered steroid refractory.

16. The method of any one of embodiments 11-15, wherein the CCL20 locked dimer comprises two CCL20 monomers, wherein at least one of the CCL20 monomers comprises SEQ ID NO: 1.

17. The method of embodiment 16, wherein the CCL20 locked dimer comprises two monomers covalently linked together, wherein each monomer comprises SEQ ID NO: 1 or an amino acid sequence with at least a 95% sequence identity to SEQ ID NO: 1 and a S64C mutation relative to SEQ ID NO:1.

18. The method of embodiment 16, wherein each monomer comprises SEQ ID NO: 1.

19. The method of embodiment 18, wherein each monomer consists of SEQ ID NO: 1.

20. A method of reducing the likelihood of a subject developing graft versus host disease (GVHD) in a subject in need thereof, the method comprising: administering a therapeutically effective amount of a composition comprising a CCL20 locked dimer (CCL20LD) to the subject to reduce the likelihood of the subject developing GVHD.

21. The method of embodiment 20, wherein the CCL20LD is administered prior to the subject receiving an allogeneic stem cell transplant.

22. The method of embodiment 20, wherein the subject has been treated with an allogeneic stem cell transplant, and wherein the subject exhibits no signs or symptoms of GVHD.

23. The method of embodiment 20, wherein the subject has been treated with an allogeneic stem cell transplant, and wherein the allogeneic stem cell transplant comprised at least one HLA mismatch to the subject, and wherein the subject exhibits no signs or symptoms of GVHD.

24. The method of any one of embodiments 20-23, wherein the CCL20 locked dimer comprises two CCL20 monomers, wherein at least one of the CCL20 monomers comprises SEQ ID NO: 1.

25. The method of embodiment 24, wherein the CCL20 locked dimer comprises two monomers covalently linked together, wherein each monomer comprises SEQ ID NO: 1 or an amino acid sequence with at least a 95% sequence identity to SEQ ID NO: 1 and a S64C mutation relative to SEQ ID NO:1.

26. The method of embodiment 25, wherein each monomer comprises SEQ ID NO: 1.

27. The method of embodiment 26, wherein each monomer consists of SEQ ID NO: 1.

28. A composition comprising SEQ ID NO: 1, linked to an XTEN polypeptide sequence.

29. The composition of embodiment 28, wherein the XTEN polypeptide is located on the C terminus of SEQ ID NO: 1 or wherein the XTEN polypeptide is located on the N terminus of the composition.

30. The method of any one of embodiments 1-27, wherein the composition is the composition of embodiment 27 or 28.

31. A method of treating ocular graft versus host disease (oGVHD) in a subject in need thereof, the method comprising: administering a therapeutically effective amount of a composition comprising a CCL20 locked dimer to the subject to treat oGVHD.

32. The method of embodiment 31, wherein the CCL20 locked dimer comprises two CCL20 monomers, wherein at least one of the CCL20 monomers comprises SEQ ID NO: 1.

33. The method of embodiment 32, wherein the CCL20 locked dimer comprises two monomers covalently linked together, wherein each monomer comprises SEQ ID NO: 1 or an amino acid sequence with at least a 95% sequence identity to SEQ ID NO: 1 and a S64C mutation relative to SEQ ID NO:1.

34. The method of embodiment 32, wherein each monomer comprises SEQ ID NO: 1.

35. The method of embodiment 32, wherein each monomer consists of SEQ ID NO: 1.

36. The method of any one of embodiments 31-35, wherein administering comprises ophthalmic topical administration.

37. The method of embodiment 36, wherein the composition comprising the CCL20 locked dimer is formulated for ophthalmic topical administration.

38. The method of embodiment 37, wherein the composition comprising the CCL20 locked dimer comprises a topical drop in saline or buffered salt solution.

### EXAMPLES

The following Examples are illustrative and should not be interpreted to limit the scope of the claimed subject matter.

### Example 1—CCL20 Locked Dimer is an Anti-Inflammatory Molecule

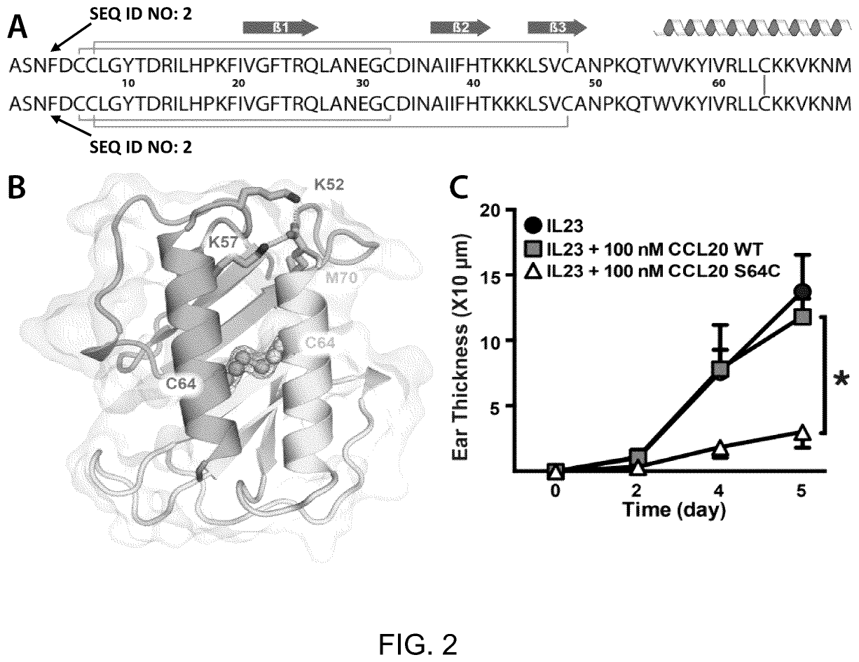

Chemokines and their G protein-coupled receptors direct the transit of immune cells, stem cells and many cancers in and out of the bone marrow, circulation and every organ and tissue in the body. Previously, the inventors designed and patented a cross-linked CCL20 dimer with the goal of blocking CCR6⁺ T cell-mediated inflammation. CCL20LD binds the CCR6 receptor with nanomolar affinity but its altered signaling properties conferred the ability to block T cell chemotaxis and prevent the development and progression of disease in a mouse model of human psoriasis (FIG. 2C) (1).

CCL20LD reverses IL-23-induced psoriatic inflammation. IL-23's role in human psoriasis and as an inducer of psoriatic disease mouse models is well established (24, 25). Using a systemic IL23-dependent mouse model that produces significant skin inflammation and joint swelling (26), the inventors recently showed that CCL20LD administration significantly reduces skin inflammation (FIG. 3A, B), joint swelling (FIG. 3C, D) and enthesitis (not shown) (2). Importantly, these data show that mice treated with CCL20LD at a daily dosage of 100 ug exhibited a significantly greater therapeutic effect on ear inflammation compared to those that received 100 μg anti-17A antibodies every other day, as measured by lower ear thickness, PSI score and decreased infiltration of neutrophils and CCR6+ T cells. The inventors also showed that stromal cells in human tendon tissue produce CCL20 upon activation with IL-1β, establishing a critical role for tenocytes in the initiation and perpetuation of psoriatic arthritis, analogous to CCL20 production by keratinocytes in psoriatic skin (2).

Taken together, these results demonstrate that an engineered CCL20 variant designed to stabilize a CXC-type dimer interface (CCL20LD) binds CCR6 and effectively blocks Th17 cell-mediated inflammation in mouse models of psoriatic disease.

### Example 2—Examining the Effect of CCL20 Locked Dimer in a Mouse Model of GVHD

The efficacy of CCL20LD in a murine model of cGVHD that reflects the clinical features cGVHD in humans (27) will be assessed. The inventors have observed Th17 expansion in the GVHD target tissues such as skin, liver, lung and gut, starting in late-stage aGVHD and early-stage cGVHD, and the progress of cGVHD pathogenesis was associated with further expansion of CCR6-expressing Th17 cells in those tissues (3, 20, 27, 28). As shown in FIG. 4, the increase in CCR6⁺IL17⁺CD4⁺ T cells in peripheral lymph nodes is associated with a dramatic increase in skin CCL20 expression (3).

Experimental design & methods. Lethal total body irradiation (TBI)-conditioned BALB/c recipients will be induced to develop cGVHD by transplanting spleen cells (1.25×10⁶) and bone marrow cells (2.5×10⁶) from MHC-mismatched C57BL/6 donors, as described in previous publications (27, 29, 30). At cGVHD onset (30 days post-HCT) recipients will be treated with intraperitoneal (IP) injection of CCL20LD (100 μg), CCL20WT (100 μg) or buffer every other day for 30 days. Each recipient will be monitored for clinical signs of GVHD, including body weight, fur status, diarrhea, and survival. At day 60, ⅔ of recipients will be used for continuous observation for up to 100 days after HCT, and ⅓ of the recipients will be used for studying tissue infiltration of T cell subsets including Th/Tc1, Th/Tc2 and Th/Tc17 as well as for evaluation of tissue cGVHD severity by histopathology and tissue collagen deposition. Similar experiments will be performed at day 100. The experiments will be repeated at least two times with a total of 20 mice per group. The recipients will include both male and female animals.

Data analysis and interpretation The clinical cGVHD severity and survival from days 30 to 100 will be compared. The tissue infiltrating T cell subsets as well as tissue cGVHD and fibrosis at day 60 and day 100 after HCT will also be compared. Statistical analysis of survival will be performed with log-rank test and statistical analysis of disease severity and infiltration difference will be by two-way ANOVA analysis between the vehicle control and treated groups as described in our recent publication (30).

Results: It is anticipated that administration of CCL20LD at cGVHD onset (30 days after HCT) will effectively prevent cGVHD progress, and the effect will be maintained after ending treatment at day 60. However, it is possible that Th17 cells that infiltrate GVHD target tissues before cGVHD onset continue to expand in the tissue to mediate cGVHD even after CCL20LD treatment. In this case, whether administration of CCL20LD early after HCT will prevent the progress of cGVHD will be tested. It is also possible that cGVHD in the CCL20LD-treated recipients will relapse after ending treatment at day 60. In this case, treatment will be extended. In both alternative scenarios, it is anticipated that subjects treated with CCL20LD will exhibit fewer symptoms of GVHD than untreated controls.

Comments: Without wishing to be bound by theory, it is hypothesized that cGVHD pathogenesis is due to CCL20-depedent recruitment of donor-type Th17 cells into the GVHD target tissues. The engineered CCL20LD protein blocks Th17 cell recruitment, subsequent expression of IL-17A, and the additional recruitment of Th17 cells by CCL20 produced by tissue cells (e.g. keratinocytes). It is anticipated that CCL20LD will exhibit a therapeutic effect in treating cGVHD, defined by one or more of the following criteria as described in the literature, e.g., 30, 31: statistically significant increase in body weight, reduction of clinical and pathological scores, and reduction of infiltrating Th17 cells and collagen deposition in the GVHD target tissues.

### Example 3—Treatment of Graft Versus Host Disease with CCL20 Locked Dimer (CCL20LD)

In one example, a subject suffering from GVHD, e.g., acute GVHD, chronic GVHD, is administered a therapeutically effective amount of a composition comprising a CCL20 locked dimer (CCL20LD). CCL20LD may suitably be administered by any route that is indicated by the particular treatment needs of the subject, e.g., oral, transdermal, percutaneous, intravenous, intramuscular, intranasal, buccal, intrathecal, intracerebral, or intrarectal routes. Signs and symptoms of the GVHD may be reduced by the administration of the CCL20LD. Treatment may be administered daily, every other day, every third day, or on a schedule as determined by the patient's progress, pursuant to a physician's decision. It is anticipated that the subject will experience an increase in body mass, appetite, or amelioration of nausea, vomiting, abdominal pain, or diarrhea, or other metrics associated with reduction in signs or symptoms of GVHD, as compared to an untreated subject. Methods of measuring reductions in signs and symptoms of GVHD are known in the art.

### Example 4—Treatment of Ocular Graft Versus Host Disease (oGVHD) with CCL20 Locked Dimer (CCL20LD)

In one example, a subject suffering from ocular GVHD (oGVHD) is administered a therapeutically effective amount of a composition comprising a CCL20 locked dimer (CCL20LD). CCL20LD may suitably be administered by any route that is indicated by the particular treatment needs of the subject, e.g., topically to the eye, intraocularly, orally, or intravenously. Signs and symptoms of the GVHD may be reduced by the administration of the CCL20LD. Treatment may be administered daily, every other day, every third day, or on a schedule as determined by the patient's progress, pursuant to a physician's decision. It is anticipated that the subject may experience an increase in quality of life associated with reduction in signs or symptoms of oGVHD as compared to an untreated subject. Methods of measuring reductions in signs and symptoms of oGVHD are known in the art, e.g., ocular surface disease index (OSDI), tear-film break-up time (TBUT), the Schirmer test, and cornea fluorescein staining and subjective screening methods for symptoms of oGVHD, e.g., dry eye, foreign body sensation, redness, epiphora, photophobia, blurred vision, and eye irritation.

### Example 5—Design of XTEN-Stabilized Version of CCL20LD that Retains Full Potency as an Inhibitor of T Cell Chemotaxis

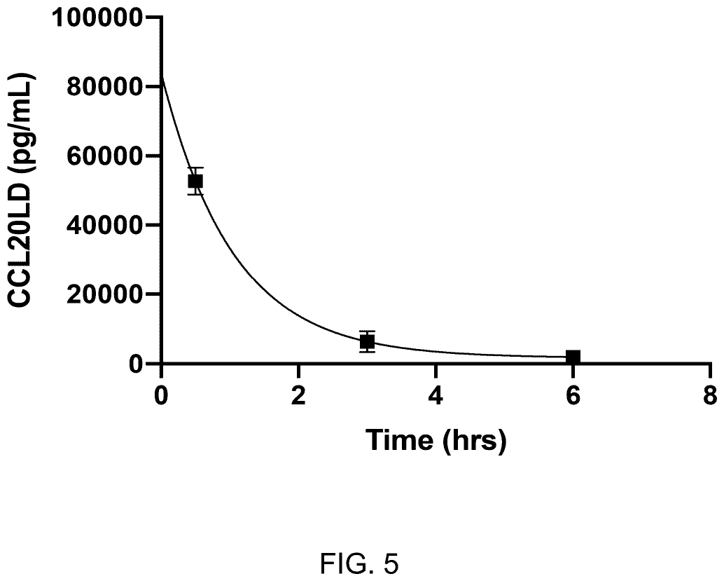

The feasibility of using CCL20LD to treat Th17-mediated inflammation has already been demonstrated in mice, with a significant reduction in the severity scores of both joint and paw inflammation in established psoriatic disease. Experiments described above will assess the efficacy of CCL20LD in a mouse model for cGVHD and lay the groundwork for additional preclinical drug development and first-in-human clinical trials. Preclinical optimization of the lead molecule is commonly required to improve known drug characteristics such as potency, bioavailability, duration, safety, and drug-like qualities. Preliminary pharmacokinetic analysis showed that CCL20LD has a relatively short half-life in mice (FIG. 5).

To lengthen the in vivo lifetime of CCL20LD (e.g., so that a pharmacologically effective therapy can be achieved with once-daily dosing), the inventors propose to employ polypeptide-based masking and half-life extension technology to enhance in vivo potency and bioavailability of CCL20LD.

Experimental Design & Methods. To enhance potency by extending the bioavailability of CCL20LD and limit the number of injections needed for in-human trials, an optimized CCL20LD with reduced clearance will be designed and tested for improved in vitrolin vivo efficacy. In one exemplary approach, the inventors will design and produce two or more CCL20LD-XTEN fusion proteins. XTEN (Amunix, San Franciso, CA) is an unstructured polypeptide composed exclusively of six amino acids, alanine, glutamate, glycine, proline, serine, and threonine, that can be added to the N- or C-terminus of a protein payload (32). Varying the XTEN sequence in size from a few hundred to >1,000 amino acids has been shown to increase the hydrodynamic radius of small protein payloads preventing renal clearance and improve the resident half-life of therapeutic proteins in vivo. Further, the length of the XTEN polypeptide is directly correlated with the resident half-life allowing fine tuning of the in vivo half-life (32-34). The DNA sequence coding for the XTEN peptide will be appended to the C-terminus of the E. coli CCL20LD expression system disclosed in U.S. Pat. No. 10,738,095 which is incorporated by reference herein in its entirety. Expression, refolding and purification protocols for CCL20-XTEN fusion proteins production will be developed to produce the gram amounts of active pharmaceutical ingredient (API) required in clinical trials.

To show that CCL20LD-XTEN is produced with the highest quality and purity and demonstrate the molecule's scale-up potential three (3) lots of at least three (3) milligrams each of CCL20LD-XTEN will be produced. An individual lot will be classified as the API derived from expression and purification of a freshly transformed bacterial cell growth. After three lots are produced, a battery of quality control (QC) testing SOPs will be used to evaluate lot-lot consistency, all of which employ methods known and accepted in the art (1, 35-37).

SDS-PAGE-5 μg samples of API from three individual lots will be analyzed by 4-20% Tris Glycine gels. Protein band intensity will be analyzed on a ChemiDoc digital imager (BioRad) and intensities calculated using vendor-supplied software.

ESI-Mass Spectrometry-1 μM sample of API from three individual lots will be analyzed on a ThermoFisher Fusion Lumos mass spectrometer.

¹H NMR-50 μM sample of API from three individual lots will be prepared in an NMR buffer optimized to give the most reproducible spectra possible.

CCR6 activation-CCL20LD biological activity will be evaluated by CCR6-mediated release of intracellular calcium using a CCR6-expressing human T cell line as previously described (1). A series of CCL20LD concentrations will be tested and EC₅₀ values will be calculated by non-linear regression fitting of the plotted data.

Inhibitory potency-Inhibition of CCR6-mediated T cell chemotaxis in response to 30 nM WT CCL20 will be measured as previously described (1). A series of CCL20LD concentrations for the lead CCL20LD molecule and each XTEN-conjugated version will be tested and IC₅₀ values will be estimated from non-linear regression fitting.

Data Analysis and Interpretation. QC testing release criteria outline follows. Any lot that fails to meet the testing standards will be classified as non-satisfactory. In this event, protocols will be reviewed and after identification of production error, a new lot will be produced and tested.

SDS-PAGE—Densitometry analysis will show the CCL20LD protein band will be ≥ 90% of total observed protein in the gel lane.

ESI-Mass Spectrometry—Analysis of intact purified protein will show the major species of each lot to have a parent ion mass within 75 ppm of the expected mass (<1.5 Da from the calculated MW).

¹H NMR—Lots will be qualitatively assessed for proper folding and the presence of identifiable fingerprint resonances (across all three lots) characteristic of properly manufactured CCL20LD.

CCR6 agonist activity-Individual lot calcium flux EC₅₀ values will be within 10% of the three lot-averaged EC₅₀ value to ensure reproducible bioactivity.

Inhibitory potency—IC₅₀ values for each XTEN-CCL20LD formulation should be 250 nM or lower (i.e., within 2.5-fold of the CCL20LD IC₅₀=100 nM.)

Alternative constructs: In some embodiments, the XTEN polypeptide will be covalently attached to the CCL20LD protein by sortase enzyme-catalyzed ligation at the C-terminus.

### Example 6—CCL20LD Reduces Pathogenic T Cell Frequency in a Model of Chronic Graft-Versus Host Disease

The inventors evaluated the effect of CCL20LD in a minor mismatch (H2Kb-B6 H2Kb-C3h.sw) murine bone marrow transfer model. The results reveal a trend of reduction in the percentage of TH17 cells in tissues affected by cGVHD (FIGS. 6 and 7A-7C).

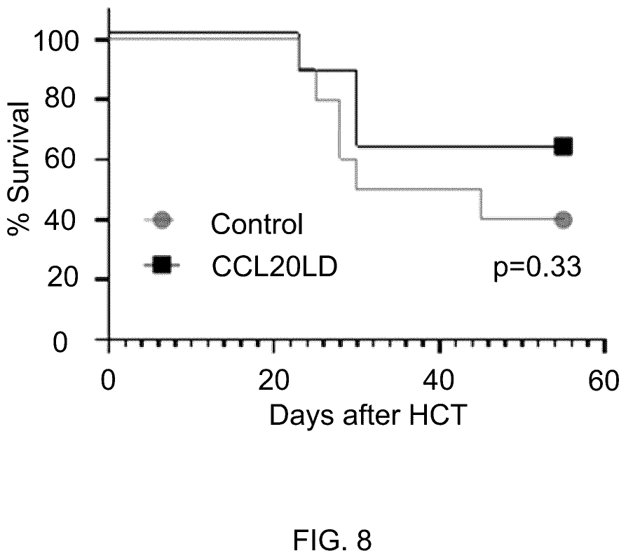

In addition, with reference to FIG. 8, the results demonstrate an increase in survival in the CCL20LD treated group, though the increase was not statistically significant. The inventors note that a survival benefit is not commonly observed in this mouse model of cGVHD. For this reason, even the small increase in survival benefit in the CCL20LD treated group is encouraging. It is well known that showing improved absolute survival in murine models of chronic GVHD is extremely difficult. This is because the normal supportive care that is so essential for humans with GVHD, such as supplemental nutrition and intensive skin care, cannot be rendered to mice. As such, an improvement in survival in the CCL20LD-treated mice, while not strictly mathematically significant in this small sample, is none-the less considered a very promising signal of efficacy of the CCL20LD treatment.

### REFERENCES

1. Getschman A E, Imai Y, Larsen O, Peterson F C, Wu X, Rosenkilde M M, Hwang S T, Volkman B F. Protein engineering of the chemokine CCL20 prevents psoriasiform dermatitis in an IL-23-dependent murine model. Proc Natl Acad Sci USA. 2017; 114 (47): 12460-5. Epub 2017 Nov. 8. doi: 10.1073/pnas.1704958114. PubMed PMID: 29109267; PMCID: PMC5703275.
2. Shi Z, Garcia-Melchor E, Wu X, Getschman A E, Nguyen M, Rowland D J, Wilson M, Sunzini F, Akbar M, Huynh M, Law T, Kundu-Raychaudhuri S K, Raychaudhuri S P, Volkman B F, Millar N L, Hwang S T. Targeting the CCR6/CCL20 axis in entheseal and cutaneous inflammation. Arthritis Rheumatol. 2021. Epub 2021 Jun. 4. doi: 10.1002/art.41882. PubMed PMID: 34081845.
3. Jin H, Ni X, Deng R, Song Q, Young J, Cassady K, Zhang M, Forman S, Martin P J, Liu Q, Zeng D. Antibodies from donor B cells perpetuate cutaneous chronic graft-versus-host disease in mice. Blood. 2016; 127 (18): 2249-60. Epub 2016 Feb. 18. doi: 10.1182/blood-2015-09-668145. PubMed PMID: 26884373; PMCID: PMC4859199.
4. Hill L, Alousi A, Kebriaei P, Mehta R, Rezvani K, Shpall E. New and emerging therapies for acute and chronic graft versus host disease. Ther Adv Hematol. 2018; 9 (1): 21-46. Epub 2018 Jan. 11. doi: 10.1177/2040620717741860. PubMed PMID: 29317998; PMCID: PMC5753923.
5. Bachier C R, Aggarwal S K, Hennegan K, Milgroom A, Francis K, Dehipawala S, Rotta M. Epidemiology and Treatment of Chronic Graft-versus-Host Disease Post-Allogeneic Hematopoietic Cell Transplantation: A US Claims Analysis. Transplant Cell Ther. 2021; 27 (6): 504 e1-e6. Epub 2021 Jun. 24. doi: 10.1016/j.jtct.2020.12.027. PubMed PMID: 34158154.
6. FDA requires warnings about increased risk of serious heart-related events, cancer, blood clots, and death for JAK inhibitors that treat certain chronic inflammatory conditions. U.S. Food and Drug Administration, 2021 Sep. 1, 2021. Report No.: Sep. 1, 2021.
7. Ratajczak P, Janin A, Peffault de Latour R, Leboeuf C, Desveaux A, Keyvanfar K, Robin M, Clave E, Douay C, Quinquenel A, Pichereau C, Bertheau P, Mary J Y, Socie G. Th17/Treg ratio in human graft-versus-host disease. Blood. 2010; 116 (7): 1165-71. Epub 2010 May 21. doi: 10.1182/blood-2009-12-255810. PubMed PMID: 20484086; PMCID: PMC3001485.
8. Reinhardt K, Foell D, Vogl T, Mezger M, Wittkowski H, Fend F, Federmann B, Gille C, Feuchtinger T, Lang P, Handgretinger R, Andreas Bethge W, Holzer U. Monocyte-induced development of Th17 cells and the release of S100 proteins are involved in the pathogenesis of graft-versus-host disease. J Immunol. 2014; 193 (7): 3355-65. Epub 2014 Sep. 5. doi: 10.4049/jimmunol. 1400983. PubMed PMID: 25187661.
9. Forcade E, Paz K, Flynn R, Griesenauer B, Amet T, Li W, Liu L, Bakoyannis G, Jiang D, Chu H W, Lobera M, Yang J, Wilkes D S, Du J, Gartlan K, Hill G R, MacDonald K P, Espada E L, Blanco P, Serody J S, Koreth J, Cutler C S, Antin J H, Soiffer R J, Ritz J, Paczesny S, Blazar B R. An activated Th17-prone T cell subset involved in chronic graft-versus-host disease sensitive to pharmacological inhibition. JCI Insight. 2017; 2 (12). Epub 2017 Jun. 15. doi: 10.1172/jci.insight.92111. PubMed PMID: 28614794; PMCID: PMC5470889.
10. Nishimori H, Maeda Y, Teshima T, Sugiyama H, Kobayashi K, Yamasuji Y, Kadohisa S, Uryu H, Takeuchi K, Tanaka T, Yoshino T, Iwakura Y, Tanimoto M. Synthetic retinoid Am80 ameliorates chronic graft-versus-host disease by down-regulating Th1 and Th17. Blood. 2012; 119 (1): 285-95. Epub 2011 Nov. 15. doi: 10.1182/blood-2011-01-332478. PubMed PMID: 22077062.
11. Meitei H T, Jadhav N, Lal G. CCR6-CCL20 axis as a therapeutic target for autoimmune diseases. Autoimmun Rev. 2021; 20 (7): 102846. Epub 2021 May 11. doi: 10.1016/j.autrev.2021.102846. PubMed PMID: 33971346.
12. Lyu M, Li Y, Hao Y, Lyu C, Huang Y, Sun B, Li H, Xue F, Liu X, Yang R. CCR6 defines a subset of activated memory T cells of Th17 potential in immune thrombocytopenia. Clin Exp Immunol. 2019; 195 (3): 345-57. Epub 2018 Nov. 8. doi: 10.1111/cei.13233. PubMed PMID: 30403287; PMCID: PMC6378379.
13. Paradowska-Gorycka A, Wajda A, Romanowska-Prochnicka K, Walczuk E, Kuca-Warnawin E, Kmiolek T, Stypinska B, Rzeszotarska E, Majewski D, Jagodzinski P P, Pawlik A. Th17/Treg-Related Transcriptional Factor Expression and Cytokine Profile in Patients With Rheumatoid Arthritis. Front Immunol. 2020; 11:572858. Epub 2020 Dec. 29. doi: 10.3389/fimmu.2020.572858. PubMed PMID: 33362761; PMCID: PMC7759671.
14. Lu G, Zhang X, Shen L, Qiao Q, Li Y, Sun J, Zhang J. CCL20 secreted from IgA1-stimulated human mesangial cells recruits inflammatory Th17 cells in IgA nephropathy. PLOS One. 2017; 12 (5): e0178352. Epub 2017 May 30. doi: 10.1371/journal.pone.0178352. PubMed PMID: 28552941; PMCID: PMC5446182.
15. Hedrick M N, Lonsdorf A S, Hwang S T, Farber J M. CCR6 as a possible therapeutic target in psoriasis. Expert Opin Ther Targets. 2010; 14 (9): 911-22. Epub 2010 Jul. 16. doi: 10.1517/14728222.2010.504716. PubMed PMID: 20629596.
16. Furue M, Furue K, Tsuji G, Nakahara T. Interleukin-17A and Keratinocytes in Psoriasis. Int J Mol Sci. 2020; 21 (4). Epub 2020 Feb. 20. doi: 10.3390/ijms21041275. PubMed PMID: 32070069; PMCID: PMC7072868.
17. van der Waart A B, van der Velden W J, van Halteren A G, Leenders M J, Feuth T, Blijlevens N M, van der Voort R, Dolstra H. Decreased levels of circulating IL17-producing CD161⁺CCR6⁺ T cells are associated with graft-versus-host disease after allogeneic stem cell transplantation. PLOS One. 2012; 7 (12): e50896. Epub 2012 Dec. 12. doi: 10.1371/journal.pone.0050896. PubMed PMID: 23226545; PMCID: PMC3514180.
18. MacDonald K P, Hill G R, Blazar B R. Chronic graft-versus-host disease: biological insights from preclinical and clinical studies. Blood. 2017; 129 (1): 13-21. Epub 2016 Nov. 9. doi: 10.1182/blood-2016-06-686618. PubMed PMID: 27821504; PMCID: PMC5216261.
19. Harper E G, Guo C, Rizzo H, Lillis J V, Kurtz S E, Skorcheva I, Purdy D, Fitch E, Iordanov M, Blauvelt A. Th17 cytokines stimulate CCL20 expression in keratinocytes in vitro and in vivo: implications for psoriasis pathogenesis. J Invest Dermatol. 2009; 129 (9): 2175-83. Epub 2009 Mar. 20. doi: 10.1038/jid.2009.65. PubMed PMID: 19295614; PMCID: PMC2892172.
20. Yi T, Chen Y, Wang L, Du G, Huang D, Zhao D, Johnston H, Young J, Todorov I, Umetsu D T, Chen L, Iwakura Y, Kandeel F, Forman S, Zeng D. Reciprocal differentiation and tissue-specific pathogenesis of Th1, Th2, and Th17 cells in graft-versus-host disease. Blood. 2009; 114 (14): 3101-12. Epub 2009 Jul. 16. doi: 10.1182/blood-2009-05-219402. PubMed PMID: 19602708; PMCID: PMC2756212.
21. Drury L J, Ziarek J J, Gravel S, Veldkamp C T, Takekoshi T, Hwang S T, Heveker N, Volkman B F, Dwinell M B. Monomeric and dimeric CXCL12 inhibit metastasis through distinct CXCR4 interactions and signaling pathways. Proc Natl Acad Sci USA. 2011; 108 (43): 17655-60. Epub 2011 Oct. 13. doi: 10.1073/pnas.1101133108. PubMed PMID: 21990345; PMCID: PMC3203819.
22. Takekoshi T, Ziarek J J, Volkman B F, Hwang S T. A locked, dimeric CXCL12 variant effectively inhibits pulmonary metastasis of CXCR4-expressing melanoma cells due to enhanced serum stability. Mol Cancer Ther. 2012; 11 (11): 2516-25. Epub 2012 Aug. 8. doi: 10.1158/1535-7163.MCT-12-0494. PubMed PMID: 22869557; PMCID: PMC3496366.
23. Veldkamp C T, Seibert C, Peterson F C, De la Cruz N B, Haugner J C, 3rd, Basnet H, Sakmar T P, Volkman B F. Structural basis of CXCR4 sulfotyrosine recognition by the chemokine SDF-1/CXCL12. Science signaling. 2008; 1 (37): ra4. Epub 2008 Sep. 19. doi: 10.1126/scisignal. 1160755. PubMed PMID: 18799424; PMCID: PMC2692298.
24. Hedrick M N, Lonsdorf A S, Shirakawa A K, Richard Lee C C, Liao F, Singh S P, Zhang H H, Grinberg A, Love P E, Hwang S T, Farber J M. CCR6 is required for IL-23-induced psoriasis-like inflammation in mice. J Clin Invest. 2009; 119 (8): 2317-29. Epub 2009 Aug. 8. PubMed PMID: 19662682.
25. Ghoreschi K, Weigert C, Rocken M. Immunopathogenesis and role of T cells in psoriasis. Clin Dermatol. 2007; 25 (6): 574-80. Epub 2007 Nov. 21. doi: 10.1016/j.clindermatol.2007.08.012. PubMed PMID: 18021895.
26. Sherlock J P, Joyce-Shaikh B, Turner S P, Chao C C, Sathe M, Grein J, Gorman D M, Bowman E P, McClanahan T K, Yearley J H, Eberl G, Buckley C D, Kastelein R A, Pierce R H, Laface D M, Cua D J. IL-23 induces spondyloarthropathy by acting on ROR-gammat+CD3+CD4−CD8− entheseal resident T cells. Nat Med. 2012; 18 (7): 1069-76. Epub 2012 Jul. 10. doi: 10.1038/nm.2817. PubMed PMID: 22772566.
27. Wu T, Young J S, Johnston H, Ni X, Deng R, Racine J, Wang M, Wang A, Todorov I, Wang J, Zeng D. Thymic damage, impaired negative selection, and development of chronic graft-versus-host disease caused by donor CD4⁺ and CD8⁺ T cells. J Immunol. 2013; 191 (1): 488-99. Epub 2013 May 28. doi: 10.4049/jimmunol. 1300657. PubMed PMID: 23709681; PMCID: PMC3746979.
28. Zeng D. Newly found arsons ignite the fire of gut GVHD. J Clin Invest. 2018; 128 (3): 897-9. Epub 2018 Jan. 30. doi: 10.1172/JCI98685. PubMed PMID: 29376890; PMCID: PMC5824851.
29. Deng R, Hurtz C, Song Q, Yue C, Xiao G, Yu H, Wu X, Muschen M, Forman S, Martin P J, Zeng D. Extrafollicular CD4 (+) T-B interactions are sufficient for inducing autoimmune-like chronic graft-versus-host disease. Nat Commun. 2017; 8 (1): 978. Epub 2017 Oct. 19. doi: 10.1038/s41467-017-00880-2. PubMed PMID: 29042531; PMCID: PMC5645449.
30. Kong X, Zeng D, Wu X, Wang B, Yang S, Song Q, Zhu Y, Salas M, Qin H, Nasri U, Haas K M, Riggs A D, Nakamura R, Martin P J, Huang A, Zeng D. Tissue-resident PSGL1loCD4+ T cells promote B cell differentiation and chronic graft-versus-host disease-associated autoimmunity. J Clin Invest. 2021; 131 (1). Epub 2020 Sep. 16. doi: 10.1172/JCI135468. PubMed PMID: 32931481; PMCID: PMC7773409.
31. Song Q, Wang X, Wu X, Qin H, Li Y, Riggs A D, Martin P J, Chen Y Z, Zeng D. Tolerogenic anti-IL-2 mAb prevents graft-versus-host disease while preserving strong graft-versus-leukemia activity. Blood. 2021; 137 (16): 2243-55. Epub 2021 Jan. 30. doi: 10.1182/blood.2020006345. PubMed PMID: 33511398; PMCID: PMC8063091.
32. Schellenberger V, Wang C W, Geething N C, Spink B J, Campbell A, To W, Scholle M D, Yin Y, Yao Y, Bogin O, Cleland J L, Silverman J, Stemmer W P. A recombinant polypeptide extends the in vivo half-life of peptides and proteins in a tunable manner. Nat Biotechnol. 2009; 27 (12): 1186-90. Epub 2009 Nov. 17. doi: 10.1038/nbt. 1588. PubMed PMID: 19915550.
33. Alters S E, McLaughlin B, Spink B, Lachinyan T, Wang C W, Podust V, Schellenberger V, Stemmer W P. GLP2-2G-XTEN: a pharmaceutical protein with improved serum half-life and efficacy in a rat Crohn's. PLOS One. 2012; 7 (11): e50630. PLOS One. 2013; 8 (6). Epub 2013 Jun. 14. doi: 10.1371/annotation/5c7138bd-5602-466d-8daf-e75e0b7d7fdb. PubMed PMID: 23762221; PMCID: PMC3677843.
34. Cleland J L, Geething N C, Moore J A, Rogers B C, Spink B J, Wang C W, Alters S E, Stemmer W P, Schellenberger V. A novel long-acting human growth hormone fusion protein (VRS-317): enhanced in vivo potency and half-life. J Pharm Sci. 2012; 101 (8): 2744-54. Epub 2012 Jun. 9. doi: 10.1002/jps.23229. PubMed PMID: 22678811; PMCID: PMC3427893.
35. Veldkamp C T, Koplinski C A, Jensen D R, Peterson F C, Smits K M, Smith B L, Johnson S K, Lettieri C, Buchholz W G, Solheim J C, Volkman B F. Production of Recombinant Chemokines and Validation of Refolding. Methods Enzymol. 2016; 570:539-65. Epub 2016 Feb. 29. doi: 10.1016/bs.mie.2015.09.031. PubMed PMID: 26921961; PMCID: PMC4811038.
36. Ziarek J J, Volkman B F. NMR in the Analysis of Functional Chemokine Interactions and Drug Discovery. Drug discovery today Technologies. 2012; 9 (4): e293-e9. Epub 2012 Nov. 21. doi: 10.1016/j.ddtec.2012.04.003. PubMed PMID: 23166561; PMCID: PMC3499625.
37. Riutta S J, Larsen O, Getschman A E, Rosenkilde M M, Hwang S T, Volkman B F.

Mutational analysis of CCL20 reveals flexibility of N-terminal amino acid composition and length. J Leukoc Biol. 2018; 104 (2): 423-34. Epub 2018 Aug. 17. doi: 10.1002/JLB.1VMA0218-049R. PubMed PMID: 30114340.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

TABLE 1
Sequences.

| SEQ | | |
|---|---|---|
| ID NO: | Description | Sequence |
| 1 | CCL20 S64C | ASNFDCCLGYTDRILHPKFIVGFTRQLANEG |
| | | CDINAIIFHTKKKLSVCANPKQTWVKYIVRL |
| | | LCKKVKNM |
| 2 | human CCL20 | ASNFDCCLGYTDRILHPKFIVGFTRQLANEG |
| | WT | CDINAIIFHTKKKLSVSANPKQTWVKYIVRL |
| | | LSKKVKNM |

1. A method of treating graft versus host disease (GVHD) in a subject in need thereof, the method comprising: administering a therapeutically effective amount of a composition comprising a CCL20 locked dimer to the subject to treat GVHD in the subject.

2. The method of claim 1, wherein the subject is suffering from acute GVHD (aGVHD).

3. The method of claim 1, wherein the subject is suffering from chronic GVHD (cGVHD).

4. The method of claim 1, wherein the subject has previously failed steroid treatments or is considered steroid refractory.

5. The method of claim 1, wherein the CCL20 locked dimer comprises two CCL20 monomers, wherein at least one of the CCL20 monomers comprises SEQ ID NO: 1.

6. The method of claim 5, wherein the CCL20 locked dimer comprises two monomers covalently linked together, wherein each monomer comprises SEQ ID NO: 1 or an amino acid sequence with at least a 95% sequence identity to SEQ ID NO:1 and a S64C mutation relative to SEQ ID NO:1.

7. The method of claim 6, wherein each monomer comprises SEQ ID NO: 1.

8. The method of claim 6, wherein each monomer consists of SEQ ID NO: 1.

9. The method of claim 1, wherein the method reduces a sign or symptom related to graft versus host disease (GVHD) selected from: anorexia, nausea, vomiting, abdominal pain, diarrhea, jaundice, oral tissue inflammation, dry mouth, punctate or generalized mucosal erythema, white striae or papules on the oral mucosa and lips, mucosal erosion-desquamation-ulceration, pain or sensitivity to spicy foods, difficulty swallowing, pain with swallowing, pharyngo-esophageal stricture, xerostomia, lichen planus, poor bolus control, pharyngeal retention, excessive mucous secretion, oral tissue inflammation, and ulceration.

10. A method of reducing the likelihood of a subject developing graft versus host disease (GVHD) in a subject in need thereof, the method comprising: administering a therapeutically effective amount of a composition comprising a CCL20 locked dimer (CCL20LD) to the subject to reduce the likelihood of the subject developing GVHD.

11. The method of claim 10, wherein the CCL20LD is administered prior to the subject receiving an allogeneic stem cell transplant.

12. The method of claim 10, wherein the subject has been treated with an allogeneic stem cell transplant, and wherein the subject exhibits no signs or symptoms of GVHD.

13. A method of treating ocular graft versus host disease (oGVHD) in a subject in need thereof, the method comprising: administering a therapeutically effective amount of a composition comprising a CCL20 locked dimer to the subject to treat oGVHD.

14. The method of claim 13, wherein the CCL20 locked dimer comprises two CCL20 monomers, wherein at least one of the CCL20 monomers comprises SEQ ID NO: 1.

15. The method of claim 14, wherein the CCL20 locked dimer comprises two monomers covalently linked together, wherein each monomer comprises SEQ ID NO: 1 or an amino acid sequence with at least a 95% sequence identity to SEQ ID NO: 1 and a S64C mutation relative to SEQ ID NO:1.

16. The method of claim 14, wherein each monomer comprises SEQ ID NO: 1.

17. The method of claim 14, wherein each monomer consists of SEQ ID NO: 1.

18. The method of claim 13, wherein administering comprises ophthalmic topical administration.

19. The method of claim 13, wherein the composition comprising the CCL20 locked dimer is formulated for ophthalmic topical administration.

20. The method of claim 19, wherein the composition comprising the CCL20 locked dimer comprises a topical drop in saline or buffered salt solution.


SEQUENCE LISTING (2 sequences)

SEQ ID NO: 1 - protein, 70 aa, synthetic construct
ASNFDCCLGYTDRILHPKFIVGFTRQLANEGCDINAIIFHTKKKLSVCANPKQTWVKYIVRLLCKKVKNM

SEQ ID NO: 2 - protein, 70 aa, Homo sapiens
ASNFDCCLGYTDRILHPKFIVGFTRQLANEGCDINAIIFHTKKKLSVSANPKQTWVKYIVRLLSKKVKNM